(19) United States
(12) Patent Application Publication
(10) Pub. No.: US 2020/0018708 A1
(43) Pub. Date: Jan. 16, 2020

(54) DETECTION OF A CONTAMINANT IN A CONDUCTING PATH FOR AN OPERATING MEDIUM

(71) Applicant: Robert Bosch GmbH, Stuttgart (DE)

(72) Inventors: Andreas Kuebler, Remshalden (DE); Michael Kern, Stuttgart (DE); Roman Haeberle, Gemmrigheim (DE); Robert Kuehner, Engelsbrand (DE)

(21) Appl. No.: 16/468,783

(22) PCT Filed: Oct. 18, 2017
(86) PCT No.: PCT/EP2017/076567

§ 371 Date: Jun. 12, 2019

(30) Foreign Application Priority Data
Dec. 12, 2016 (DE) 10 2016 224 656.9

(51) Int. Cl. G01N 21/94; G01N 21/85; G01N 21/03; G01N 33/28

(52) U.S. Cl. CPC G01N 21/94; G01N 33/2829; G01N 21/0332; G01N 21/85

(57) ABSTRACT

A method (100) for detecting a contaminant (3) in an operating medium (2), which is conducted in a machine or apparatus (1) in a conducting path, wherein inspection light (22) is radiated (130) through at least one optical measurement location (15) within the conducting path, which inspection light comprises at least one wavelength for which the absorption coefficient of the operating medium (2) differs from the absorption coefficient of the contaminant (3), and wherein the optical absorption A of the inspection light (22) in the operating medium (2) is measured (140), wherein additionally the temperature T of the operating medium (2) at the optical measurement location (15) is determined (120). A device (20) for carrying out a method (100) according to one of claims 1 to 9, comprising at least one light source (21) and at least one detector (23) for the inspection light (22), wherein additionally at least one flow-through cuvette (24) is provided, which can be integrated into the conducting path for the operating medium (2) and through which the inspection light (22) can be radiated and upstream of which, the flow direction of the operating medium, (2), a temperature sensor (25) and/or a heating and/or cooling element (26) is connected.

### BACKGROUND OF THE INVENTION

The present invention relates to a method for detecting a contaminant in a conducting path for an operating fluid, in particular for monitoring transfers of media in an appliance which conducts both the operating fluid and the contaminant.

High-pressure pumps for diesel injection systems have hitherto been lubricated by the diesel fuel itself. Since contaminants in the diesel fuel can lead to the destruction of the high-pressure pump and consequently to major engine damage, there is, in particular for fuel-critical markets, a demand for high-pressure pumps which utilize the oil circuit of the engine for their lubrication and are thus insensitive to contaminants of the diesel fuel.

If the high-pressure pump conducts both diesel fuel and engine oil, it is important for the transfer rate between these two media not to exceed a level specified by the customer. Contamination of the diesel fuel with engine oil can impair the exhaust-gas characteristics, such that limit values in this regard are possibly missed. Conversely, contamination of the engine oil with diesel fuel can have the effect that the engine spins in uncontrolled fashion.

With the development of a high-pressure pump and of the seals contained therein, the transfer rate cannot be predicted exactly, but must be determined by measurements on prototypes. Changes to the prototype and measurements of the transfer rate are performed in succession in multiple iterative steps, until ultimately the final version of the high-pressure pump with a transfer rate according to specifications has been developed.

Here, the duration of the measurements is a significant time factor. For each measurement, the high-pressure pump must be operated at least until it exhibits a media transfer which lies above the detection limit of the measuring method used. Further time is required for the measurement itself. In this respect, there is a conflict of aims between the detection sensitivity and the lengthiness of the measurement. If the measuring method is relatively insensitive, the high-pressure pump must run for a very long time for each measurement. For very sensitive measurements, such as for example atomic emission spectrometry (ICP-OES), it is, by contrast, generally necessary to send samples to external laboratories.

DE 103 60 563 A1 has disclosed a method with which the state of contamination of liquids in a circuit can be monitored continuously, that is to say in real time, by optical means. This method is designed as an inexpensive solution for household appliances.

### SUMMARY OF THE INVENTION

In the context of the invention, a method for detecting a contaminant in an operating fluid which is conducted in a machine or apparatus in a conducting path has been developed. Here, interrogation light is radiated through at least one optical measurement location within the conducting path. The interrogation light comprises at least a wavelength for which the absorption coefficient of the operating fluid differs from the absorption coefficient of the contaminant. The optical absorption A of the interrogation light in the operating fluid is measured.

According to the invention, the temperature T of the operating fluid at the optical measurement location is additionally determined.

A determination of the temperature T is to be understood to mean any form of acquisition of knowledge regarding the value of the temperature T. This includes both passive acquisition of knowledge by direct or indirect measurement and the setting of the temperature T to a known value by means of open-loop or closed-loop control.

It has been identified that, specifically in the case of the distinction of atoms or molecules of the operating fluid, on the one hand, and of the contaminant, on the other hand, by means of electron absorption spectrometry, the exact knowledge of the temperature T is definitive of the concentration C above which the contaminant in the operating fluid can be detected. Both the width of the spectral lines and the height of absorption peaks are highly temperature-dependent. A cause of this is that, with increasing temperature, the viscosity of the operating fluid or of the contaminant changes, such that the absorption peaks widen and at the same time decrease in height. With sufficiently exact knowledge of the temperature T, the measurement results however remain informative.

The conducting path may in particular be part of a circuit in which the operating fluid is conducted in the machine or apparatus. It is for example then possible to follow, in real-time, how the contaminant gradually increases in concentration in the operating fluid.

In a particularly advantageous embodiment of the invention, the temperature T of the operating fluid at the optical measurement location is controlled in closed-loop fashion to a predefined setpoint value T_{S}. This firstly makes measurements performed successively in terms of time at the same temperature T=T_{S} to be quantitatively compared. Secondly, the quantitative determination of the concentration C of the contaminant is also made significantly easier by means of the comparison with reference samples.

The temperature T₁ of the operating fluid is advantageously measured upstream of the optical measurement location in the flow direction. If this measurement is performed close enough to the optical measurement location, T₁ is a good approximate value for the true temperature T of the operating fluid at the optical measurement location. The measurement upstream of the optical measurement location has the advantage that the optical measurement location through which the interrogation light is radiated contains no temperature sensor that could influence the interrogation light.

It is particularly advantageously the case that, in addition, the temperature T₂ of the operating fluid is additionally measured downstream of the optical measurement location in the flow direction. Then, T₂ can be offset using T₁ in order to arrive at an even more exact approximation for the temperature T that is not measured directly at the optical measurement location.

The measurement and the closed-loop control of the temperature T are not mutually exclusive. Firstly, the closed-loop control requires feedback of the temperature T, for example in the form of the measured temperature T₁ and/or of the measured temperature T₂. Secondly, by means of the measurement, the closed-loop control quality of the closed-loop control can be monitored, and the influence of closed-loop control errors can be more effectively eliminated from the measurement results.

For example, from the absorption A of the operating fluid at the temperature T, the absorption A′ that the operating fluid would exhibit at a different temperature T′ can be evaluated. Such an evaluation is based on the fact that the physical relationships which, in the event of a change in the temperature, lead to a change and widening of spectral lines are quantitatively known. It is thus for example possible to model the extent to which the absorption changes during the transition from the temperature T′ to the temperature T, and this change can be inverted on the basis of the model.

In a particularly advantageous embodiment of the invention, from the comparison of the absorption A of the operating fluid with the absorption A* of at least one reference sample containing a known concentration of the contaminant, the concentration C of the contaminant in the operating fluid is evaluated. For example, reference samples may be produced which contain a mixture of the operating fluid with the contaminant, wherein the contaminant is present in concentrations C of 1, 5, 10, 15 and 20 ppm respectively. The absorption A of the interrogation light in each of the reference samples can be measured and stored in a calibration table. If the absorption A of the interrogation light is subsequently determined at the optical measurement location in the machine or apparatus, and if it lies between the absorptions A determined for two reference samples, the concentration C of the contaminant can be restricted to the range between the concentrations C that correspond to the two reference samples. The concentration C may also be determined even more exactly, for example by interpolation of the intermediate value ranges between the reference samples.

Such a quantitative determination of concentration is made significantly easier if the absorption A is measured in the reference samples at exactly the same temperature as the absorption at the optical measurement location in the machine or apparatus. The values for the absorption A are then directly comparable. It is thus advantageously the case for all measurements that the temperature T is controlled in closed-loop fashion to the same setpoint value T_{S}. For optimum accuracy in the determination of concentration, the temperature T should advantageously be kept constant within ±0.1° C.

In a further particularly advantageous embodiment of the invention, the contaminant is mixed with a contrast agent, wherein the absorption coefficient of the contrast agent for the interrogation light deviates to a greater extent from the absorption coefficient of the operating fluid for the interrogation light than the absorption coefficient of the contaminant alone for the interrogation light. In this way, the signal caused by a contaminant which is chemically related or similar to the operating fluid in the absorption measurement can be greatly intensified.

For example, the contrast between engine oil and diesel fuel for interrogation light of a wavelength of 650 nm can be greatly increased by virtue of one of the two substances being pigmented with Sudan Blue 673. In principle, use may for example also be made of a Sudan Red or a Sudan Yellow. A Sudan Blue however has the major advantage that its absorption spectrum has no similarity to the absorption spectrum of the engine oil itself even if the engine oil ages. As a result of coking and other ageing processes, the engine oil changes color such that it absorbs interrogation light at the same wavelengths as a Sudan Red or Sudan Yellow admixed as contrast agent. If it is merely of importance to detect the start of a media transfer in the first place, this is not an issue. By contrast, if it is sought to quantitatively detect the media transfer, the result can be falsified if the engine oil is misinterpreted as contrast agent, and accordingly a much higher concentration C of the engine oil is inferred.

The machine or apparatus advantageously comprises at least one appliance which, during operation, is flowed through both by the operating fluid and by the contaminant on respectively nominally mutually separate paths. This is the main usage situation, in which it is desirable to quantitatively detect a media transfer of the contaminant into the operating fluid. For example, the appliance may be a pump for the operating fluid, which pump is lubricated with the contaminant.

As discussed in the introduction, such a demand exists for example in the case of high-pressure pumps for diesel injection systems which are lubricated with engine oil. Therefore, in a further particularly advantageous embodiment of the invention, the operating fluid is an engine fuel and the contaminant is a lubricant, or vice versa (that is to say the lubricant is the operating fluid, and the engine fuel is the contaminant).

The invention also relates to a device for carrying out the method. Said device comprises at least one light source and at least one detector for the interrogation light.

According to the invention, in addition, at least one throughflow cuvette is provided which can be integrated into the conducting path for the operating fluid and through which the interrogation light can be radiated and upstream of which, in the flow direction of the operating fluid, there are positioned a temperature sensor and/or a heating and/or cooling element.

The throughflow cuvette is in particular constructed such that, during operation in the conducting path, it is always completely filled with the operating fluid, and such that no bubbles form in the operating fluid. The boundary surfaces that the interrogation light passes through proceeding from the ambient air on the path into a first wall of the throughflow cuvette, into the operating fluid, into a second wall of the throughflow cuvette and finally back into the ambient air are then always the same. The light intensity registered by the detector is then, in the case of constant intensity of the light source, dependent only on the absorption A of the interrogation light in the operating fluid in the throughflow cuvette.

If a temperature sensor is provided, then the temperature T₁ of the operating fluid before it enters the cuvette can be registered. It is then possible at least to monitor the extent to which said temperature T₁ remains constant, and/or the measurement result for the absorption A can be corrected by the influence of a change in the temperature T₁. Using a heating and/or cooling element, the temperature T₁ can be actively set to a desired value.

It is advantageous if, in addition, a temperature sensor is positioned downstream of the throughflow cuvette in the flow direction of the operating fluid. Said temperature sensor can register the temperature T₂ of the operating fluid after it emerges from the throughflow cuvette. In conjunction with the temperature T₁ of the operating fluid before it enters the throughflow cuvette, the temperature T prevailing within the throughflow cuvette can be determined more exactly.

In a particularly advantageous embodiment of the invention, a closed-loop controller is provided which is designed to control the temperature T of the operating fluid in closed-loop fashion to a predefined setpoint value T_{S} by applying a manipulated variable to the heating and/or cooling element. The feedback of the temperature T into the closed-loop controller may be performed for example in the form of the temperature T₁ of the operating fluid before it enters the throughflow cuvette, the temperature T₂ of the operating fluid after it emerges from the throughflow cuvette, or else in the form of an approximate value for the temperature T formed from the temperatures T₁ and T₂.

In a further particularly advantageous embodiment of the invention, the detector is part of a spectrometer. By means of a spectrometer, the wavelength of the interrogation light can be set particularly exactly. In this way, particularly good selectivity can be achieved, in particular if the operating medium and contaminant are chemically similar or related. For example, the introduction of engine oil into diesel fuel can be measured with an accuracy in the ppm range.

Also possible and expedient, however, are other applications in which relatively low detection sensitivity is sufficient and accordingly a miniaturized version of the device can be used. For example, the device may be arranged in the fuel feed line from the tank to the engine and monitor whether the fuel line is carrying only the correct fuel type. In the event of misfueling, it is for example possible for the fuel feed line to be shut off, such that no incorrect fuel passes into the engine. In this way, major engine damage is prevented. Only the cleaning of the tank and of the fuel feed line is necessary in order to make the vehicle operational again.

Further measures which improve the invention will be presented in more detail below together with the description of the preferred exemplary embodiments of the invention on the basis of figures.

### BRIEF DESCRIPTION OF THE DRAWINGS

In the figures:

FIG. 1 shows an exemplary embodiment of a device according to the invention, integrated into an apparatus;

FIG. 2a shows a flow diagram of the method;

FIG. 2b shows a variant of the determination of the temperature T;

FIG. 3 shows a profile with respect to time of the measured absorption A during an endurance running test;

FIG. 4 shows a comparison of the measurement results of the method with other measurement methods.

### DETAILED DESCRIPTION

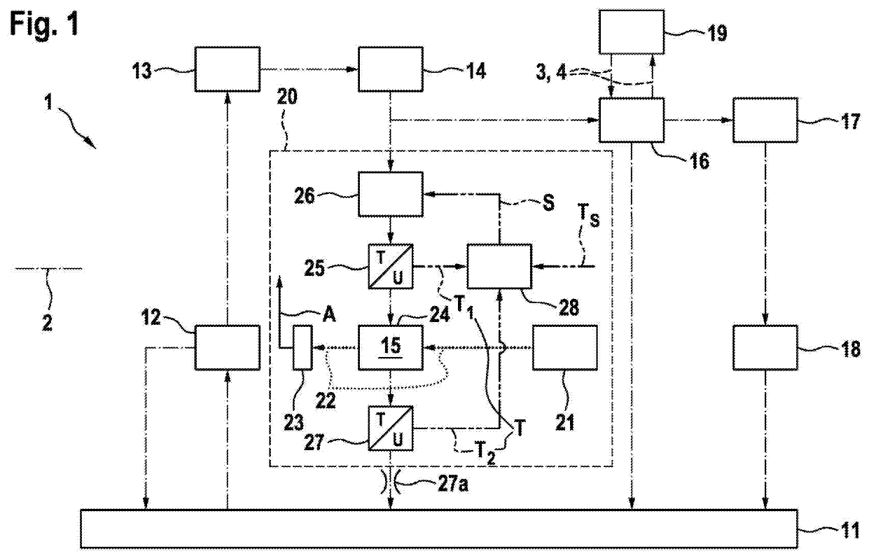

FIG. 1 schematically shows an exemplary embodiment of the device 20, which, in an apparatus 1, is integrated into the circuit for diesel fuel 2 as operating fluid. The apparatus 1 serves for measuring the extent to which a media transfer of the engine oil 3 into the diesel fuel 2 occurs in a high-pressure pump 16 for diesel fuel 2, which pump is lubricated with engine oil 3.

The diesel fuel 2 is, from a tank 11 via an intermediate reservoir 12, brought to a positive pressure of 3.6 bar, and conducted through an EPC filter 14, by a predelivery pump 13. The conveying rate is approximately 170 l/h.

Proceeding from the EPC filter 14, a first part of the diesel fuel 2 is fed to the high-pressure pump 16 to be tested. The high-pressure pump 16 feeds a fuel rail 17 to which injectors 18 are connected. Since the apparatus 1 serves only for testing the high-pressure pump 16, the diesel fuel 2 conveyed through the injectors 18 is not burned in a combustion chamber, but is conducted back into the tank 11. That fraction of the diesel fuel 2 which is discharged from the high-pressure pump 16 as an excess quantity is also conducted back into the tank 11.

Proceeding from the EPC filter 14, a second part of the diesel fuel 2, approximately 5 l/h, is conducted through the device 20. In the device 20, the diesel fuel 2 firstly passes through the combined cooling and heating device 26. In the cooling and heating device 26, the diesel fuel 2 is firstly cooled by thermal contact with cooling water, which is at a temperature of 10° C., and is subsequently heated by means of a heater, which can be electronically controlled in closed-loop fashion, to the desired temperature. The temperature T₁ attained here is measured by means of a first temperature sensor 25.

The diesel fuel 2 then passes the measurement location 15 in the throughflow cuvette 24 of the device 20 and, there, interrogation light 22 from the light source 21 is radiated through said diesel fuel. The absorption A of the interrogation light 22 is measured by means of the detector 23. The throughflow cuvette has an internal diameter of 10 mm.

The light source 21 and the detector 23 for the interrogation light 22 are parts of a spectrometer, the internal construction of which is not otherwise illustrated in any more detail in FIG. 1. It is a commercially available spectrometer which is originally designed for receiving the diesel fuel 2 in enclosed cuvettes for off-line measurements. The cuvette carrier provided for holding the cuvettes has been replaced by an adapted version which carries the throughflow cuvette 24 and which comprises leadthroughs for conducting the diesel fuel 2 into the throughflow cuvette 24 and for discharging the diesel fuel 2 from the throughflow cuvette 24.

After exiting the throughflow cuvette 24, the diesel fuel 2 passes a second temperature sensor 27 and is expanded by a throttle 27a into the tank 11. The throttle 27a has the effect that a positive pressure of between 1.5 and 2 bar exists in the throughflow cuvette 24, such that no bubbles form therein.

The high-pressure pump 16 is lubricated with the engine oil 3 which acts as a contaminant in the diesel fuel 2. The engine oil 3 is mixed with a Sudan Blue 4 as contrast agent. The mixture of engine oil 3 and Sudan Blue 4 circulates with a positive pressure of 1.5 bar between the lubricant tank 19 and the high-pressure pump 16.

The unattainable ideal situation is that, in the high-pressure pump 16, the diesel fuel 2 and the engine oil 3 are conducted on mutually completely separate paths and do not mix with one another. Owing to the high pressures and inevitable tolerances, a media transfer of the engine oil 3 into the diesel fuel 2 cannot be entirely avoided, but rather can merely be minimized to such an extent that the customer specification in this regard is met. The apparatus 11 illustrated in FIG. 1 is designed to quantitatively measure the media transfer of the engine oil 3 into the diesel fuel 2 in an endurance running test of the high-pressure pump 16. With the engine oil 3, a corresponding fraction of Sudan Blue 4 is at the same time also introduced into the diesel fuel 2. This Sudan Blue 4 has a different absorption coefficient for the interrogation light 22, and thus changes the absorption A registered by the detector 23.

It has hitherto only been possible to measure the media transfer offline, that is to say it has been necessary for a sample of the diesel fuel 2 to be extracted, and placed in a cuvette into a UVVIS spectrometer or even sent to an external laboratory, at particular time intervals. By contrast to this, the measurement setup shown in FIG. 1 performs an online measurement, that is to say the presence of Sudan Blue 4 in the diesel fuel 2 is immediately indicated as soon as the detection limit in this regard is overshot.

It is thus possible for the endurance running test of the high-pressure pump 16 to be immediately terminated as soon as it is found that the media transfer of the engine oil 3 into the diesel fuel 2 lies above the customer specification. The high-pressure pump 16 can then be correspondingly improved, and subjected to a new endurance running test. Thus, altogether considerably less time is required per iteration, and the development of the high-pressure pump 16 can consequently be considerably accelerated.

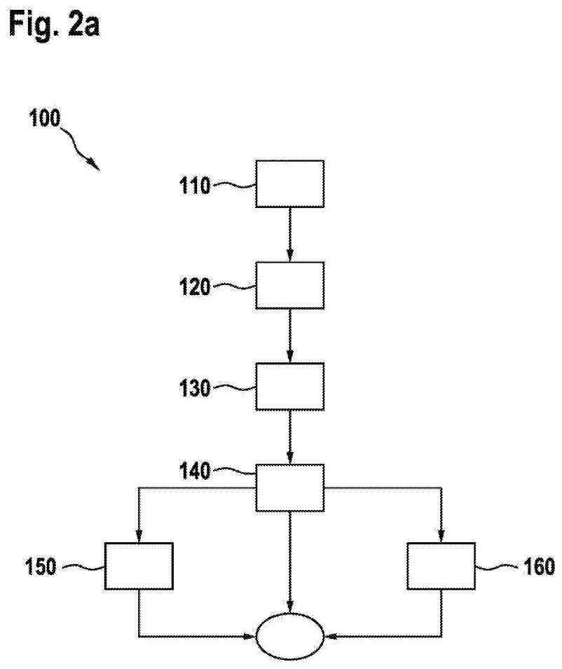

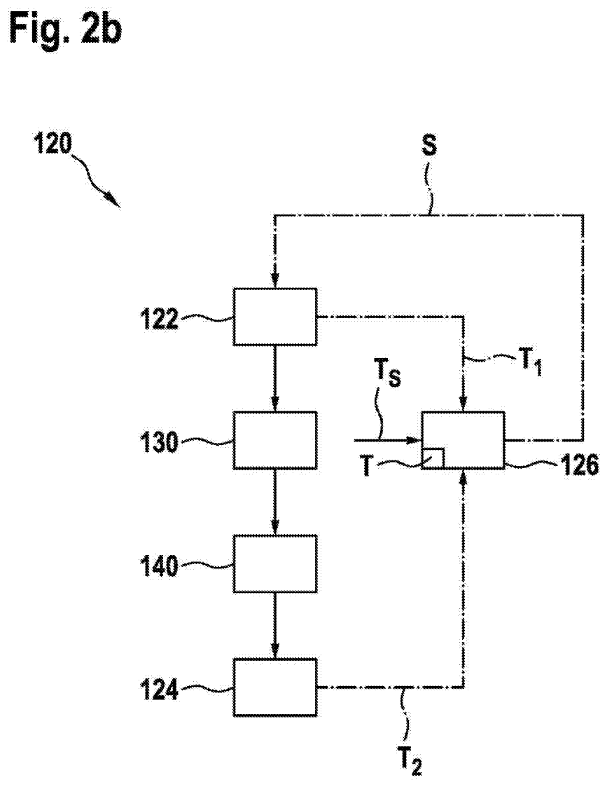

FIG. 2a shows a flow diagram of the method 100. In step 110, the engine oil 3 is mixed with the Sudan Blue 4 as contrast agent. In step 120, the temperature T of the diesel fuel 2 is determined before, in step 130, the interrogation light 22 is radiated through said diesel fuel, and in step 140, the absorption A of the interrogation light in the diesel fuel 2 is determined. This absorption A may then be retained as an end result. It is however also possible, in step 150, from the absorption A, to evaluate the absorption A′ that the diesel fuel would exhibit at a different temperature T′, for example in order to establish comparability with measurements performed on reference samples at the temperature T′. Such a comparison of the absorption A with the absorption A* of at least one reference sample which contains a known concentration C of the engine oil 3 may for example be performed in step 160.

FIG. 2b shows a variant of the determination 120 of the temperature T. In step 122, the temperature T₁ of the diesel fuel 2 upstream of the throughflow cuvette 24 in the flow direction is measured. In step 130, the interrogation light 22 is radiated through the diesel fuel 2 in the throughflow cuvette 24, and the absorption A of the interrogation light 22 is registered by the detector 23 in step 140. In step 124, the temperature T₂ of the diesel fuel 2 downstream of the throughflow cuvette 24 in the flow direction is measured.

In step 126, the temperatures T₁ and T₂ are, in the context of the active closed-loop temperature control, offset to give the temperature T of the diesel fuel 2 in the throughflow cuvette 24. A manipulated variable S is determined from the difference between this temperature T and the setpoint value T_{S}. The diesel fuel 2 is heated or cooled in accordance with the value of the manipulated variable S.

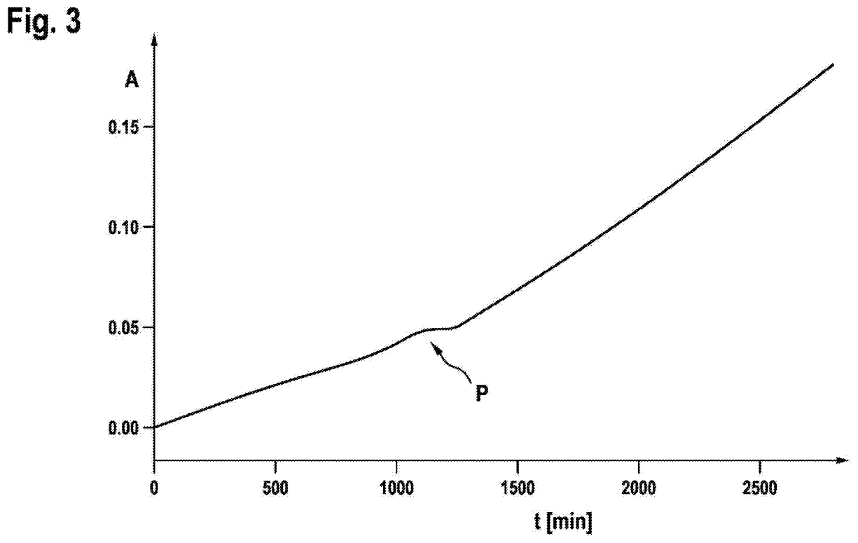

FIG. 3 shows an exemplary measurement result of an endurance running test on the apparatus 11 shown in FIG. 1. The absorption A for interrogation light 22 of a wavelength of 650 nm is plotted. At this wavelength, the diesel fuel 2 exhibits scarcely any absorption, whereas the Sudan Blue 4 exhibits particularly good absorption.

Already a few minutes after the start of the test, such a quantity of Sudan Blue 4 has collected in the diesel fuel 2 that the absorption A begins to increase approximately linearly. According to the previous prior art, up to 100 hours of continuous running of the high-pressure pump 16 were necessary before the media transfer was able to be detected.

The plateau P shows a standstill phase of the apparatus 11, in which a modification was made to the high-pressure pump 16 for testing purposes. The apparatus 11 subsequently resumes operation. The fact that the absorption A then rises with a faster rate with respect to time is an indicator for the fact that the modification made for test purposes did not yield the desired success. The rate with respect to time of the media transfer of engine oil 3 into the diesel fuel 2 has increased, rather than decreasing as hoped.

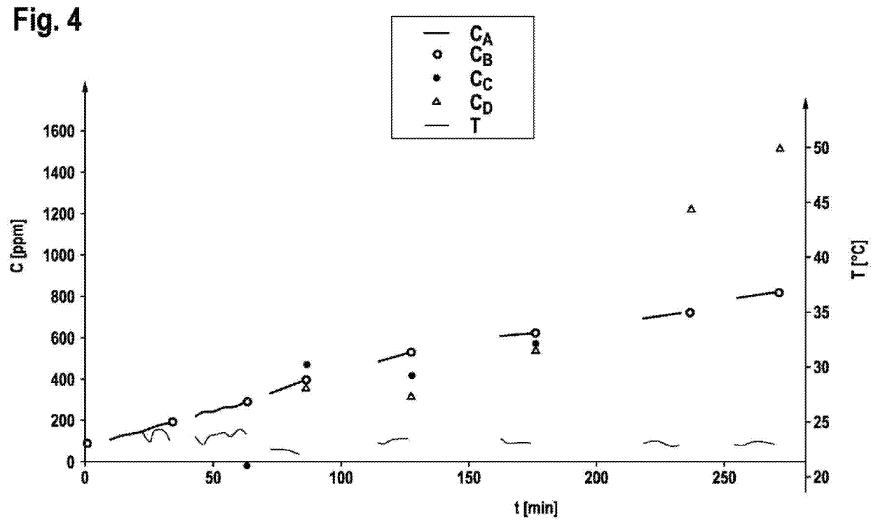

FIG. 4 shows a validation of the method 100. During a further endurance running test on the apparatus 11, the concentration C of an engine oil 3 pigmented with Sudan Blue 4 in the diesel fuel 2 was measured during a number of time intervals (curve portions CA). At the same time, in each case, the temperature T in the throughflow cuvette 24 was also determined (curve portions T).

For comparison, at the end of each time interval, that is to say at the end of each of the curve portions C_{A} in terms of time, samples of the contaminated diesel fuel 2 were extracted. These samples were tested offline, specifically using a conventional UVVIS spectrometer (measurement points C_{B}) and by optical emission spectrometry with an inductively coupled plasma (ICP). Here, on the one hand, a Zn standard was used (measurement points C_{C}), and on the other hand, a Ca standard was used (measurement points C_{D}).

The measurement points C_{B} each closely correspond to the values at the end of the curve portions C_{A}. It is thus shown that, in the transition from offline measurements with closed cuvettes to online measurement with the throughflow cuvette 24, no systematic error has been introduced into the UVVIS measurement. The altogether approximately linear increase of the concentration C of engine oil 3 in the diesel fuel 2 corresponds to an approximately constant leakage rate in the high-pressure pump 16, and is thus also physically plausible.

By contrast, the ICP measurements exhibit intense scatter about the linear increase with considerable step changes in the gradients between the measurement points. The high-pressure pump 16 does not exhibit such behavior in reality. In particular, it is not plausible that the concentration C of the engine oil 3 in the diesel fuel 2 decreases again in the time period between t=80 min and t=130 min, as per the ICP measurements. Once engine oil 3 has passed into the diesel fuel 2, it cannot escape from there again. The measurement according to the invention is thus simultaneously better, faster and cheaper than ICP measurements.

The online measurement according to the invention has further advantages in relation to the offline UVVIS measurement. Since no manual handling is required, sources of errors resulting from incorrect sample extraction and errors in handling are eliminated. Personnel are not contaminated with diesel fuel 2 and engine oil 3. It is not the case that a new single-use cuvette is required for each measurement. Furthermore, the quantity of diesel fuel 2 conducted in the circuit is not reduced as a result of sample extraction.

1. A method (100) for detecting a contaminant (3) in an operating fluid (2) which is conducted in a machine or apparatus (1) in a conducting path, the method comprising
radiating (130) interrogation light (22). which has at least a wavelength for which an absorption coefficient of the operating fluid (2) differs from an absorption coefficient of the contaminant (3), through (130) through at least one optical measurement location (15) within the conducting path,
measuring (140) an optical absorption A of the interrogation light (22) in the operating fluid (2),
determining (120) a temperature T of the operating fluid (2) at the optical measurement location (15), and
controlling in closed-loop fashion (126) the temperature T of the operating fluid (2) at the optical measurement location (15) to a predefined setpoint value T_{S}.

2. The method (100) as claimed in claim 1, characterized in that the a temperature T₁ of the operating fluid (2) upstream of the optical measurement location (15) in a flow direction is measured (122).

3. The method (100) as claimed in claim 2, characterized in that, in addition, a temperature T₂ of the operating fluid (2) downstream of the optical measurement location (15) in the flow direction is measured (124).

4. The method (100) as claimed in claim 1, characterized in that, from the absorption A of the operating fluid (2) at the temperature T, an absorption A′ that the operating fluid (2) would exhibit at a different temperature T′ is evaluated (150).

5. The method (100) as claimed in claim 1, characterized in that, from a comparison of the absorption A of the operating fluid (2) with an absorption A* of at least one reference sample containing a known concentration C of the contaminant (3), a concentration C of the contaminant (3) in the operating fluid (2) is evaluated (160).

6. The method (100) as claimed in claim 1, characterized in that the contaminant (3) is mixed (110) with a contrast agent (4), wherein an absorption coefficient of the contrast agent (4) for the interrogation light (22) deviates to a greater extent from the absorption coefficient of the operating fluid (2) for the interrogation light (22) than the absorption coefficient of the contaminant (3) alone for the interrogation light (22).

7. The method (100) as claimed in claim 1, characterized in that the machine or apparatus (1) comprises at least one appliance (16) which, during operation, is flowed through both by the operating fluid (2) and by the contaminant (3) on respectively nominally mutually separate paths.

8. The method (100) as claimed in claim 1, characterized in that the operating fluid (2) is an engine fuel, and the contaminant (3) is a lubricant, and/or in that the operating fluid (2) is a lubricant and the contaminant (3) is an engine fuel.

9. A device (20) for carrying out a method (100) as claimed in claim 1, comprising
at least one light source (21),
at least one detector (23) for the interrogation light (22),
at least one throughflow cuvette (24) integrated into the conducting path for the operating fluid (2) and through which the interrogation light (22) can be radiated,
upstream of the throughflow cuvette (24), in the flow direction of the operating fluid (2), a temperature sensor (25) and/or a heating and/or cooling element (26), and
a closed-loop controller (28) configured to control the temperature T of the operating fluid (2) in closed-loop fashion to a predefined setpoint value T_{S} by applying a manipulated variable S to the heating and/or cooling element (26).

10. The device (20) as claimed in claim 9, characterized in that, in addition, a temperature sensor (27) is positioned downstream of the throughflow cuvette (24) in the flow direction of the operating fluid (2).

11. The device (20) as claimed in claim 9, characterized in that the detector (23) is part of a spectrometer.
